(19) United States
(12) Patent Application Publication
(10) Pub. No.: US 2019/0010440 A1
(43) Pub. Date: Jan. 10, 2019

(54) CONTINUOUS FERMENTATION DEVICE

(71) Applicant: CJ Cheiljedang Corporation, Seoul (KR)

(72) Inventors: Young Ho HONG, Suwon-si (KR); Hwa Jeong LEE, Suwon-si (KR); Hong Ki KIM, Incheon (KR); Won Bae ROH, Incheon (KR); Sang Kwan JUNG, Incheon (KR); Seung Won PARK, Yongin-si (KR); Seong Jun CHO, Seoul (KR)

(73) Assignee: CJ Cheiljedang Corporation, Seoul (KR)

(21) Appl. No.: 16/065,726

(22) PCT Filed: Oct. 11, 2016
(86) PCT No.: PCT/KR2016/011379

§ 371 Date: Jun. 22, 2018

(30) Foreign Application Priority Data
Dec. 24, 2015 (KR) 10-2015-0186520

(51) Int. Cl. C12M 3/00; A23N 17/00; B01F 13/00; B01F 13/10; B01F 3/04; C12M 3/06; C12M 1/00; C12M 1/26; B65G 15/22; F24F 3/16

(52) U.S. Cl. CPC C12M 23/44; A23N 17/00; B01F 13/0013; B01F 13/1016; B01F 3/04241; B01F 2003/04319; C12M 29/06; C12M 33/00; B65G 15/22; F24F 3/16; B01F 2003/04361; C12M 27/16

(57) ABSTRACT

The present disclosure relates to a continuous fermentation device that can ferment objects continuously fed thereinto while conveying the objects. The present disclosure provides a continuous fermentation device comprising a plurality of fermentation modules, each including: a conveyor belt, which transports objects that are continuously fed thereinto to be fermented from one side to the opposite side thereof; agitators, which is located on the conveyor belt; and an air diffuser, which is located on one side of the conveyor belt, in which the continuously fed objects to be fermented are fermented while being passed through the fermentation modules. According to the present disclosure, feeding of raw materials to be fermented, fermenting the raw materials, and discharging of the fermented materials are continuously performed so that it is possible to significantly reduce the processing time, thereby reducing the operation time of the production process. Accordingly, it is possible to achieve an increase in competitiveness, such as improved productivity, reduction of investment cost, etc., in the development of a method of mass production through solid-state fermentation, thereby innovatively reducing the production cost of the product.

### TECHNICAL FIELD

The present disclosure relates to a fermentation device, and more specifically, to a continuous fermentation device which can perform fermentation of continuously fed objects to be fermented while transporting the objects.

### BACKGROUND ART

As a source of vegetable proteins for livestock, raw grain materials or by-products which remain after the extraction of active ingredients from the raw grain materials are mainly utilized as raw materials for feed. Typical examples of such vegetable proteins are soybean meal, corn gluten, etc. Soybean meal is a dehulled and defatted component of soybean meal and is currently used as various kinds of feeds of livestock, such as pigs, chickens, beef cattle, etc. Currently, soybean meal has problems in that it has a lower protein content compared to animal proteins, insufficient contents of some vitamins, minerals, and unknown growth factors (UGF), and also contains various kinds of anti-nutritional factors (ANFs), thus decreasing digestibility when used as feed. In particular, since anti-nutritional factors such as trypsin inhibitor can decrease digestibility in young livestock and thereby inhibit their growth, the amount of soybean meal to be added to feed has been limited.

To solve the above problems, studies have been performed for the purpose of decreasing the content of anti-nutritional factors while improving digestibility through fermentation using microorganisms, and soybean meal with improved quality is currently used for various livestock feeds both home and abroad. A representative example is soybean meal.

A representative method for preparing fermented soybean meal is as follows. A beneficial microorganism is introduced into soybean meal and the soybean meal is stored in a batch fermenter for a certain period of time. The soybean meal fermented by the beneficial microorganism is dried and crushed, and used as a main source of vegetable proteins to be added in the compound feed.

The main production facilities for preparing the fermented soybean meal may be largely classified into a high-temperature steam facility capable of lowering the population of general microorganisms contaminating soybean meal, a solid fermentation facility for performing solid fermentation by mixing soybean meal with a beneficial microorganism, and a drying facility for drying the fermented soybean meal after fermentation, and a crushing facility for crushing the fermented soybean meal to a size to be used as feed. The most important part requiring technological advance in the process of preparing the fermented soybean meal is the part of the solid fermentation facility. Based on the presumption that beneficial microorganisms will have the same effect and the same fermentation time, it is speculated that the effect and efficiency of the solid fermentation equipment in the manufacturing facility of the fermented soybean meal can realize a high quality product and reduction of manufacturing costs.

Basically, the solid fermenter for fermented soybean meal may vary depending on the kind of the beneficial microorganism. In a case where an aerobic microorganism is used, temperature and air permeability may be considered as major factors. For effective control of temperature and ventilation, temperature is mostly controlled within the batch fermenter.

The batch fermenter as disclosed in Patent Document 1 below is mainly used due to its excellent fermentability and simple operation technique. However, in the case of mass production in the feed industry, the requirement of the number of fermenters and the size of the fermenters are a source of major investment costs.

As shown in FIG. 18, the conventional batch fermentation device performs fermentation as follows. Raw materials (objects to be fermented) fed into the input unit 1 are distributed and fed into a plurality of fermentation modules (A) by undergoing steaming and cooling processes in steaming unit 2 and cooling unit 3, and the seed microorganism cultured in the cultivation unit 4 is introduced into a plurality of fermentation modules (A) to ferment the objects to be fermented. Additionally, a plurality of odor-processing units (6a, 6b, 6c, and 6d) for treating odors generated during the fermentation process must be provided in each fermentation device (A), and the completed objects to be fermented are sucked through the suction unit 5 connected to each fermentation device (A) through a pipe and transferred to a drying unit. However, the conventional batch fermentation device has disadvantages in that, due to the characteristics of the batch fermentation, cleaning time for removing contaminants in the fermentation device, time for feeding the strain and the raw materials, and time for discharging the fermentation product are required; the pipes may be clogged during the suction of the fermented objects to be fermented; and additionally, the productivity of the final product per fermenter is very low because each of these processes is intermittently performed in each fermentation device (A).

Accordingly, there is a need for the development of a fermenter required for solid fermentation of soybean meal and other grains in a cost-effective manner. Additionally, there is a need for the development of a continuous fermentation device so as to improve the productivity of the batch fermenter.

(Patent Document 1) KR 2015-0008265 A

### DISCLOSURE

### Technical Problem

Under these circumstances, the present disclosure is contrived based on the conventional problems described above so as to solve the problems thereof. Accordingly, an object of the present disclosure is to provide a continuous fermentation device which enables continuous fermentation of objects to be fermented so that feeding of a raw material, performing fermentation, and discharging of a fermentation product, etc. in one facility, thereby preventing problems that may occur during the operation owing to the simplified process and facility and reducing the costs for constituting the facility by reducing manufacturing facilities.

### Technical Solution

To achieve the above object, the present disclosure provides a continuous fermentation device including a plurality of fermentation modules, each fermentation module including:

a conveyor belt, which transports objects to be fermented that are continuously fed thereinto from one side to the opposite side thereof;

agitators, which are located on the conveyor belt; and

air diffusers, which are located on one side of the conveyor belt, wherein the continuously fed objects to be fermented are fermented while being passed through the fermentation modules and are continuously discharged.

The plurality of fermentation modules are preferably in a stacked form where one side and the opposite side of each conveyor belt are connected to each other, and the continuously fed objects to be fermented are continuously fermented while being passed through the plurality of fermentation modules.

The plurality of conveyor belts of the plurality of fermentation modules preferably form a stack of two rows, and each of the plurality of fermentation modules further includes a belt connection part, which connects one side and the opposite side of each of the conveyor belts in each row, and the continuously fed objects to be fermented are continuously fermented while being passed through the fermentation modules.

Each of the agitators preferably includes:

a shaft, which is located on the conveyor belt;

a plurality of support frames, wherein each support frame radially extends from one side and the opposite side of the outer circumference of the shaft;

connecting frames, wherein each connecting frame connects the ends of the support frames located at each of the opposite sides of the shaft; and

a plurality of agitating frames, wherein each agitating frame connects the connecting frames on one side and the opposite side.

Each of the agitators preferably agitates the objects to be fermented, which are transported on the conveyor belt as each of the agitating frames moves, as the shaft rotates.

The continuous fermentation device preferably includes agitation wings, in which each agitation wing is located on one side of the outer circumference of the plurality of agitating frames in the longitudinal direction.

The shaft is preferably height-adjustable.

The air diffuser preferably includes:

a main supply pipe, which is located across the top side of the conveyor belt on one side of the conveyor belt; and

distribution pipes, wherein each distribution pipe extends from one side of the main supply pipe and is located in the longitudinal direction of the top side of the conveyor belt, and a plurality of apertures are formed around the periphery thereof.

The apertures are preferably formed in at least two rows and the objects to be fermented are supplied with air through the apertures.

Each of the distribution pipes preferably injects air inside the objects to be fermented when the objects to be fermented are transported on the conveyor belt.

Each row formed by the apertures is preferably arranged at a predetermined angle from the center of the distribution pipes.

The apertures are preferably arranged in three rows on one side of the outer circumference of the distribution pipes 320, in which each row formed by the apertures is arranged with an angular difference of from 60 degrees or greater to 90 degrees or less from the center of the distribution pipes.

The continuous fermentation device preferably further includes guides, which have a predetermined height and are located in the longitudinal direction on both sides of the conveyor belt.

The continuous fermentation device preferably further includes a conveyor belt which transports the objects to be fermented that are continuously fermented and discharged to a drying unit.

The continuous fermentation device preferably further includes:

a plurality of air conditioning devices, which are provided on one side of each of the conveyor belt for suction of odors generated during the fermentation process of the objects to be fermented; and

odor-processing units, which are connected to the plurality of air conditioning devices,

in which the odors sucked from the plurality of air conditioning devices are integrated and processed in the odor-processing units.

### Advantageous Effects of the Invention

As described above, the continuous fermentation device according to the present disclosure enables continuous performance of feeding of a raw material, performing fermentation, discharging of a fermentation product, etc. in one facility, and as a result, the processing time can be significantly reduced, thereby reducing the operation time of the production process. Accordingly, the continuous fermentation device of the present disclosure can improve business competitiveness by reducing investment costs, improving productivity, etc. in the development of mass production through solid fermentation, thereby having the effect of significantly reducing the manufacturing costs of products manufactured therefrom.

### BRIEF DESCRIPTION OF DRAWINGS

FIG. 1 is an overall perspective view of a continuous fermentation device according to an embodiment of the present disclosure.

FIG. 2 is a side view of a continuous fermentation device according to an embodiment of the present disclosure.

FIG. 3 is an overall perspective view of a continuous fermentation device according to another embodiment of the present disclosure.

FIG. 4 is a side view of a continuous fermentation device according to another embodiment of the present disclosure.

FIG. 5 is a perspective view illustrating the configuration of an agitator in detail.

FIG. 6 is a perspective view illustrating the configuration of an air diffuser in detail.

FIGS. 7A and 7B are cross-sectional views taken along the line A-A′ in FIG. 6, illustrating the arrangement of the apertures and the air injection angles.

FIG. 8 is a plan view of the conveyor belt portion viewed from above.

FIG. 9 shows one side of a cross-section taken along the line B-B′ in FIG. 8.

FIGS. 10 to 13 show the comparison results with respect to fermentation effects between the conventional batch fermentation device and a continuous fermentation device according to the present disclosure: a graph on the number of live microorganisms (FIG. 10), a graph on water content (FIG. 11), a graph on crude proteins (CP) (FIG. 12), and SDS-PAGE results (FIG. 13), respectively.

FIGS. 14 to 16 show the comparison results with respect to fermentation effects between the conventional batch fermentation device and a continuous fermentation device according to the present disclosure: a graph on the number of live microorganisms (FIG. 14), a graph on a graph on crude proteins (CP) (FIG. 15), and water content (FIG. 16), respectively.

FIG. 17 shows an overall fermentation process of objects to be fermented using the continuous fermentation device according to the present disclosure.

FIG. 18 shows a fermentation process of objects to be fermented according to a conventional batch fermentation device.

### BEST MODE

The above objects, features, and other advantages of the present disclosure will become more apparent by describing in detail preferred embodiments of the present disclosure with reference to the accompanying drawings. During this process, the thicknesses of the lines and the sizes of the components shown in the drawings may be exaggerated for clarity and convenience of explanation. Additionally, the following terms are defined in consideration of the functions in the present disclosure, which may vary depending on the intentions or customs of the user or operator. Therefore, the definitions of these terms must be described based on the contents throughout this specification.

Furthermore, the embodiments described are provided for illustrative purposes of explanation and are not intended to limit the technical scope of the present disclosure.

Each component of the continuous fermentation device of the present disclosure may be used as an integral unit or as an independent unit, as needed. In addition, some components of the continuous fermentation device may be omitted depending on the forms of usage.

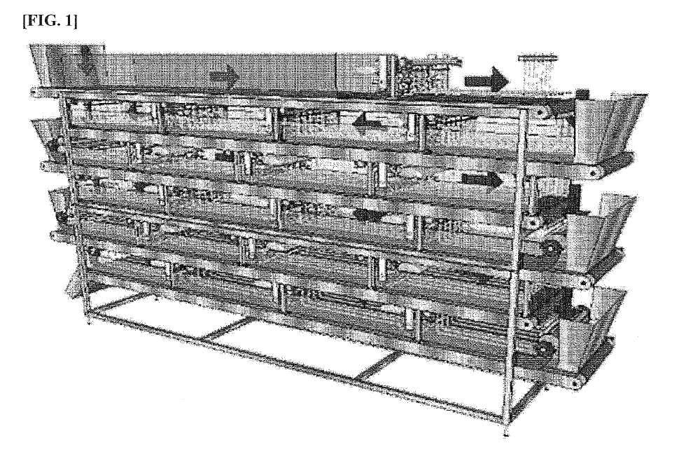

Hereinafter, a continuous fermentation device according to an embodiment of the present disclosure will be described in detail with reference to accompanying FIGS. 1 to 17.

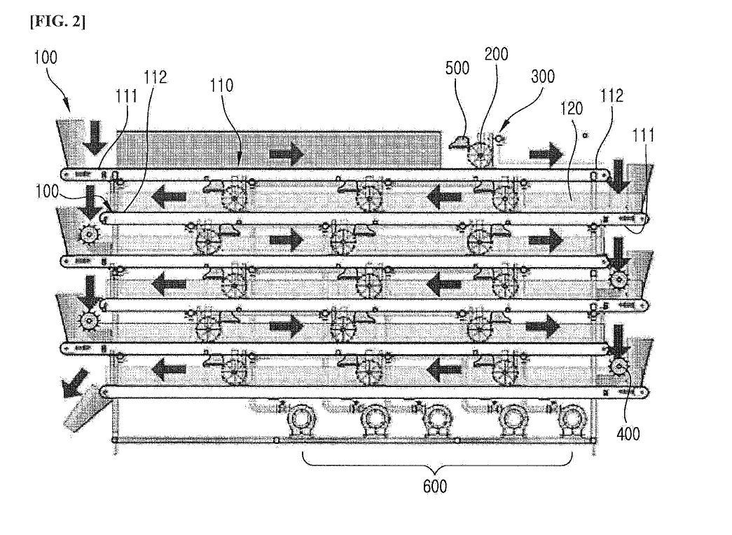

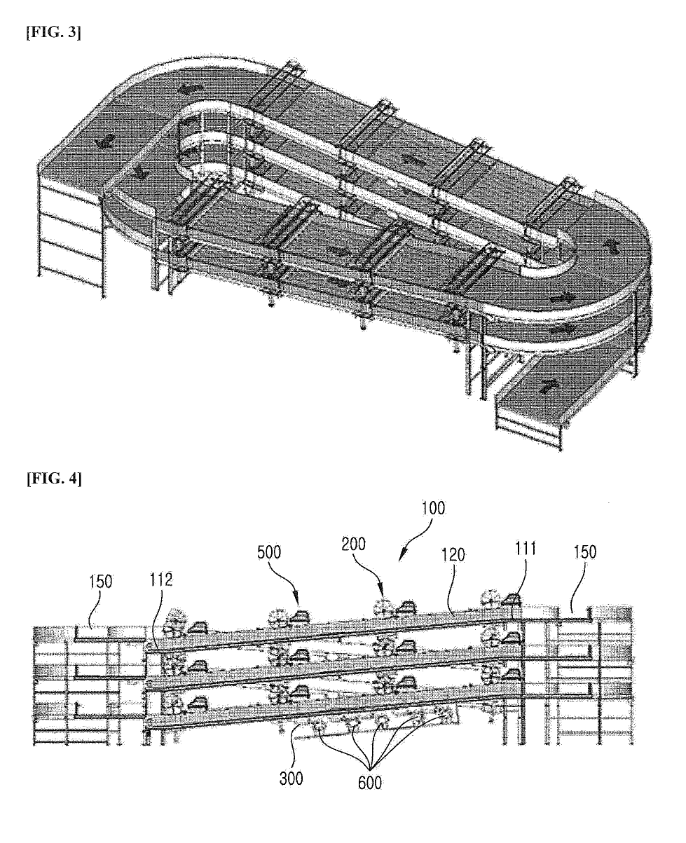

As shown in FIGS. 2 and 4, the continuous fermentation device of the present disclosure may include fermentation modules 100, where each fermentation modules 100 is provided with a conveyor belt 110, agitators 200, air diffusers 300, and guides 120; and as further shown in FIGS. 1 and 3, the continuous fermentation device of the present disclosure may be configured in a form where a plurality of fermentation modules 100 are stacked, whereby the installation space can be efficiently utilized.

The conveyor belt 110 is located in a predetermined longitudinal direction so that objects to be fermented arriving at one side 111 of the conveyor belt 110 are transported to be discharged through the opposite side 112.

The agitators 200 are located on the conveyor belt 110 and agitate the objects to be fermented which are transported by the conveyor belt 110.

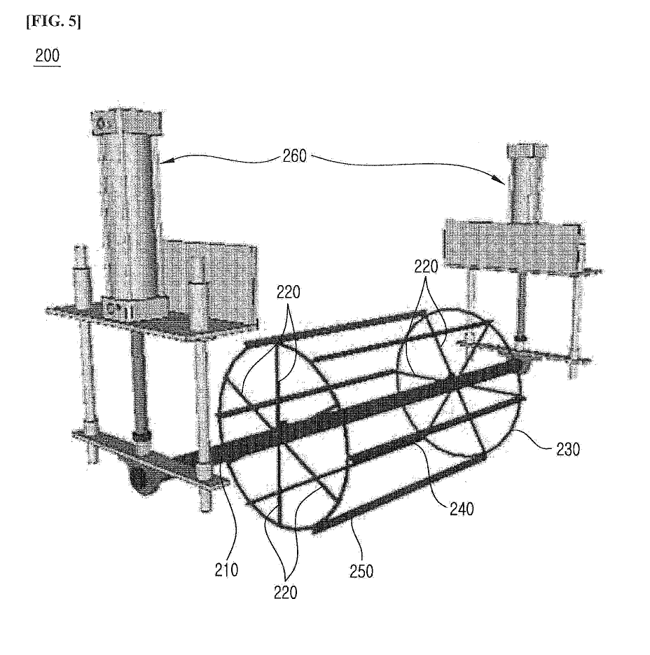

Describing the configuration of the agitators 200 in more detail by referring to FIG. 5, the agitators 200 may include a shaft 210, support frames 220, connecting frames 230, agitating frames 240, and cylinders 260.

The shaft 210 is located being spaced a predetermined distance upward across the transport direction of the conveyor belt 110.

Each of the plurality of support frames 220 radially extends from one side and the other side of the shaft 210, and each connecting frame 230 connects each end of the support frames 220.

Each agitating frame 240 is located across each connecting frame 230 to act upon rotation of the shaft 210, and the objects to be fermented that are transported on the conveyor belt 110 are agitated by the agitating frames 240.

The agitating frames 240 may be provided with an agitation wing 250 located along the longitudinal direction thereof, and as such, the area of contact with the objects to be fermented can be increased, thereby enabling a more effective agitation.

Additionally, the cylinders 260 connected to both sides of the shaft 210 allow the up-and-down movement of the shaft, and thereby, the agitation height can be adjusted according to the height of the objects to be fermented that are transported.

When the temperature of the objects to be fermented rises above the appropriate fermentation temperature by the heat generated during the fermentation, the agitators 200 agitate the objects to be fermented and thereby lower the temperature of the objects so that an appropriate temperature can be maintained.

Each air diffuser 300 is located on one side of the conveyor belt 110 and supplies air to the objects to be fermented.

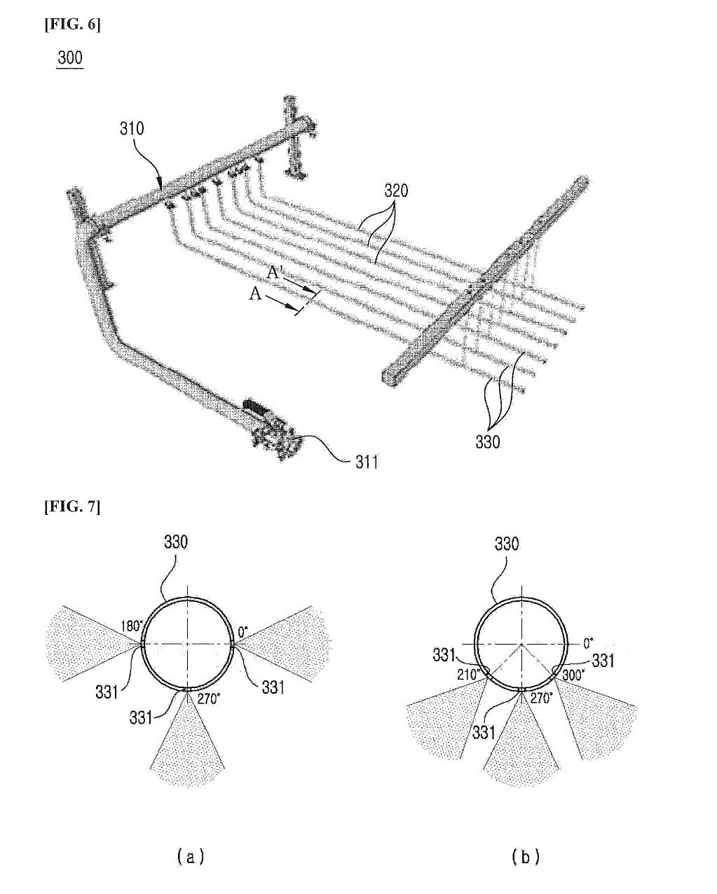

Explaining the configuration of each air diffuser 300 in more detail by referring to FIG. 6, each air diffuser 300 may include a main supply pipe 310 and distribution pipes 320.

The main supply pipe 310, being extended on the conveyor belt 110, is located across the conveyor belt 110, and air is supplied thereto through an air supply unit 600 connected to one side thereof. The amount of air being supplied from the air supply unit 600 into the main supply pipe 310 can be controlled using an air-controlling means such as a valve 311, etc., disposed on the main supply pipe 310.

Each distribution pipe 320 is connected to the main supply pipe 310 and extended toward the transport direction of conveyor belt 110 so as to have a certain length. The distribution pipe 320 may be provided in a plurality according to the width of the conveyor belt 110, and a plurality of apertures 331 are formed around the periphery thereof so that the air introduced through the main supply pipe 310 can be distributed through a plurality of distribution pipes 320 and supplied through the apertures 331 to the objects to be fermented being transported on the conveyor belt 110.

Specifically, the objects to be fermented are stacked up to a certain height and transported on the conveyor, and as the objects to be fermented are transported, each distribution pipe 320 becomes disposed inside of the stacked objects to be fermented, thereby enabling oxygen supply into the inside of the stacked objects to be fermented.

Additionally, the plurality of apertures 331 may be disposed to be formed in two or more rows. Furthermore, referring to FIGS. 7A and 7B, effective ventilation to the objects to be fermented can be achieved by pre-setting the intervals between the rows formed by the apertures 331 and the angle of the air being injected from the apertures 331 before air injection, according to the amount of the objects to be fermented. In addition, as shown in FIG. 7, when the apertures 331 of the distribution pipe 320 are formed in three rows, the intervals between the rows of the apertures 331 may be arranged to be formed at 90 degrees (FIG. 7A) or 60 degrees (FIG. 7B), respectively, from one side of the distribution pipe 320. This is because, in the fermentation of a solid material such as soybean, unlike in a liquid state, the air supply may encounter resistance in a solid state, and thus, the number of the apertures 331 and the intervals between the apertures are important for uniform air delivery to the objects to be fermented. That is, when the apertures 331 are formed in only one row and the pressure for the air to be supplied is significantly increased, the air may be delivered to the entirety of the objects to be fermented. However, in this case, the objects to be fermented located near the apertures will be over-supplied with air, which causes drying rather than fermentation, thus making it difficult to achieve uniform fermentation over the entirety of the objects to be fermented. Accordingly, uniform fermentation can be achieved by supplying air to the entirety of the objects to be fermented by disposing the apertures 331 in two or more rows so that the intervals therebetween are arranged at a predetermined angle from the center.

Additionally, each distribution pipe 320 at one end portion thereof is provided with a cap capable of opening and closing of the distribution pipe 320, and thus, the inside of each distribution pipe 320 can be cleaned by easily opening and closing the cap.

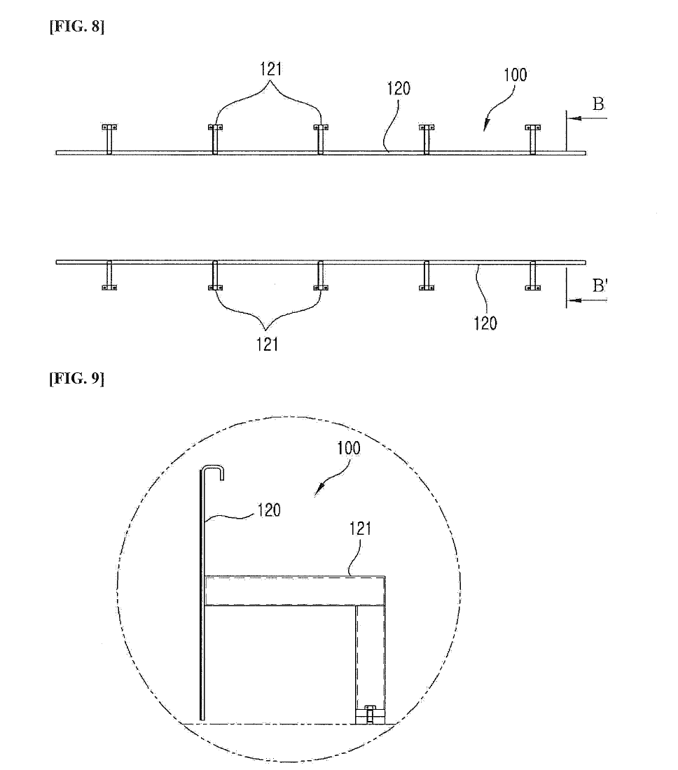

Guides 120 are located in the longitudinal direction on both sides of the conveyor belt 110, and referring further to FIG. 8, the guides 120 have a predetermined height on both sides of the conveyor belt 110, and supporting units 121 that support the guides 120 may be located at one side of each of the guides 120.

That is, the objects to be fermented transported on the conveyor belt 110 are transported while being passed through between the guides 120. When the objects to be fermented are transported on the conveyor belt 110, a phenomenon occurs in which the objects to be fermented are spread on both sides. The falling-off of these objects to be fermented from both sides of the conveyor belt 110 may be prevented by allowing extra space on both sides. However, in such case, the width of the conveyor belt 110 will become greater, thus lowering utilization of the space. Meanwhile, by providing the guide 120 on both sides of the conveyor belt 110, the spreading phenomenon of the objects to be fermented on the conveyor belt 110 and the resulting falling-off of the objects can be prevented and the width of the conveyor belt can also be minimized.

Additionally, on the top side of each conveyor belt 110, a plurality of air conditioning devices 500 for removing odors generated during fermentation of the objects to be fermented are installed. Each of the air conditioning devices 500 is connected to a single odor-processing unit 60, and thus there is an advantage in that the odors collected by suction in each air conditioning device 500 can be treated in the single odor-processing unit 60.

In an embodiment, a case where a plurality of fermentation modules 100 are stacked as described above is explained as follows, again referring to FIGS. 1 and 2. A plurality of fermentation modules 100 are stacked in a row and one side 111 and the opposite side 112 of each fermentation module 100, specifically, one side 111 and the other side 112 of each conveyor belt 110 are disposed in an alternating manner. That is, the fermentation is performed in such a manner that the objects to be fermented fed into one side 111 of the conveyor belt 110 are transported into the opposite side 112 and discharged, and the discharged objects to be fermented are again transported from one side 111 of the conveyor belt 110 located on a lower part thereof to the opposite side 112 thereof (see arrows in FIGS. 1 and 2).

Additionally, a hopper that guides the objects to be fermented, which are discharged from a conveyor belt 110 and fed into the conveyor belt 110 on a lower location thereof, and a pulverizing unit 400 which pulverizes the objects to be fermented may be located between each of the fermentation modules 100. Additionally, the air diffusers 300 are connected to each other and disposed as such, and air can be supplied from the air supply unit 600 located furthest to the bottom to the inter-connected air diffusers 300.

Additionally, in another embodiment, a case is explained where a plurality of fermentation modules 100 are stacked as described above, focusing on the difference compared to the above embodiment as follows, again referring to FIGS. 3 and 4. A plurality of fermentation modules 100 are stacked in two rows, and sides 111 and the opposite sides 112 of fermentation modules 100 located on different rows from each other are disposed to be connected by a belt connection part 150. The shape of the belt connection part 150 is not limited, but it may be in a curved shape for smooth transport of the objects to be fermented. Accordingly, the fed objects to be fermented are sequentially transported from a conveyor belt 110 in one row to a conveyor belt 110 in another row through the belt connection part 150 in an approximately elliptical shape, and thereby the objects to be fermented are fermented (see arrows in FIGS. 3 and 4).

Additionally, the conveyor belt 110′ is located connecting the opposite side of the fermentation module 100 in the final position to a drying unit, and the fermentation products, which are continuously discharged upon completion of fermentation, are transported to the drying unit (not shown).

Accordingly, the continuous fermentation device of the present disclosure can effectively transport and dry a fermentation product being continuously discharged, and enables smooth transport and drying of the fermentation product without clogging of pipes, etc., unlike the conventional batch fermentation device utilizing a suction method.

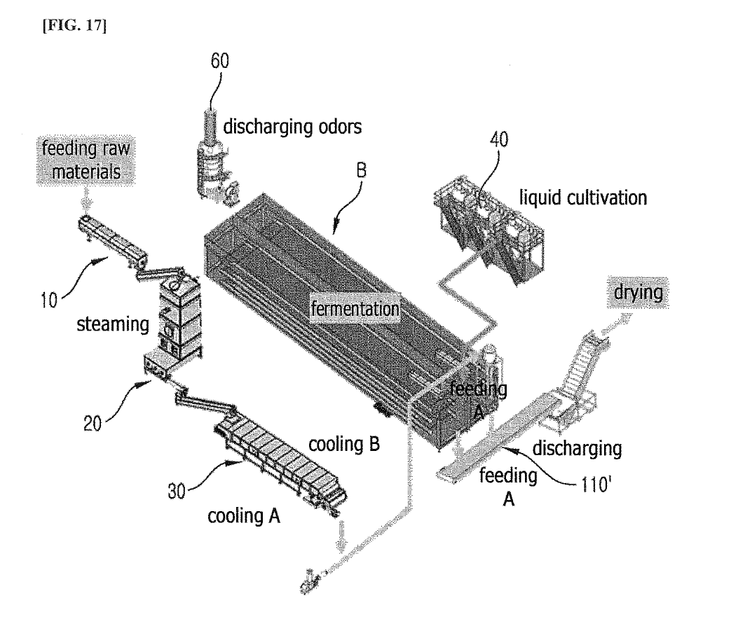

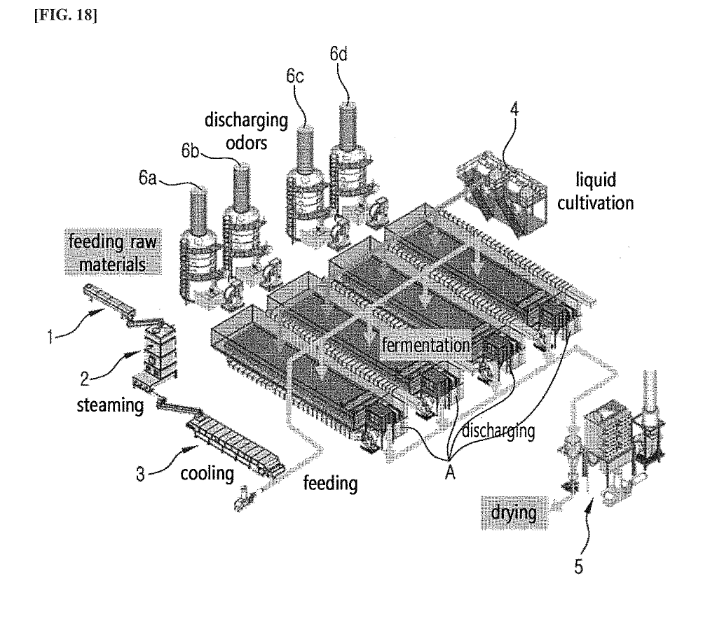

Hereinafter, again referring to FIGS. 1 to 17, the continuous fermentation device according to the present disclosure is described as an experimental example to explain the difference from the conventional batch fermentation device. To confirm the differences in fermentability and quality with the existing batch fermentation device, solid fermentation was performed by applying the continuous fermentation device according to the present disclosure. As shown in FIG. 17, the raw material (i.e., the objects to be fermented) introduced through the feeding unit 10, was fed into a fermentation device (B) consisting of a plurality of fermentation modules 100 after undergoing steaming and cooling processes in steaming unit 20 and cooling unit 30, the seed microorganism cultured in a cultivation unit 40 was added thereto and the objects to be fermented were continuously fermented, and the fermentation product being discharged upon completion of fermentation was transported to a drying unit through the conveyor belt 110′ and dried.

For this experiment, soybean meal was used as the raw material (i.e., the objects to be fermented) and the amount of soybean meal used was 60 kg to 90 kg based on the raw material. For the effective fermentation of the raw material (i.e., soybean meal), water was added (water content: at a 43% level), and then, steaming was performed to sterilize the soybean meal to which the water was added. During the steaming process, the average temperature of the soybean meal was maintained at 70° C. to 90° C. for 20 minutes. After the steaming process, the soybean meal underwent a cooling process and the seed microorganism was inoculated so as to perform the solid fermentation. As the seed microorganism, Plantarum K2G, a subspecies of Bacillus amyloliquefaciens, was used. For the stable growth of the seed microorganism, the seed microorganism was inoculated at a concentration of 10% relative to the weight of the raw material so as to secure the total number of the microorganism of 1×10⁷ cfu/mL or greater at the initial stage. The cultivation of the seed microorganism was activated in GYP medium (glucose 10.0 g, yeast extract 8.0 g, and polypeptone 2.0 g (pH 7.0)).

The objects to be fermented were fed to a height of 20 cm as a standard and samples were collected at 4-hour intervals for confirming the growth process of the seed microorganism and the change in quality of the fermentation product. The changes in the viable cell count (cfu/g), water content (%) of the fermentation product, and crude protein content (%) of each sample were measured.

For the replacement of the conventional batch fermentation device, it is extremely important that the same quality of the fermentation product under the same conditions be ensured. In the conventional batch fermentation, the fermentation of a seed microorganism is completed within 24 hours and the quality (viable cell count: 1×10⁹ cfu/g or greater, final water content: 32% or less, and increase of crude protein: 6% or greater). As such, to achieve the above quality (improvement of an increase rate of the crude protein), the control of the temperature of the fermentation product and air permeability for oxygen supply were optimized through the agitators 200 and air diffusers 300. That is, to further facilitate the permeability of oxygen to the objects to be fermented as described above, the distribution pipes 320 of the air diffusers 300 were inserted into the objects to be fermented, whereby the air permeability can be improved and the change in the overall quality of the fermentation product can be minimized.

After fermentation, the temperature of the objects to be fermented was controlled as follows while observing the change in temperature of the objects to be fermented. When the temperature of the fermentation product was increased to a certain temperature (45° C.) or higher, the agitators 200 were operated, and the temperature of the objects to be fermented was induced to decrease through the contact of the objects to be fermented with air by these agitators 200, and thereby the temperature change of the objects to be fermented from 47° C. to 39° C. was confirmed. In addition, the temperature change by the agitators 200 alone was confirmed and the effect of air diffusion during the agitation was confirmed.

As shown in Table 1 below, when the agitators 200 were operated alone, the temperature change before and after the agitation was at a level of about 3° C., whereas when the air diffusers 300 were additionally applied, the temperature change was about 8° C. Accordingly, it was confirmed that the agitation and air diffusion using both the agitators 200 and air diffusers 300 in the case of high heat generation during solid fermentation was more effective in controlling the temperature change of the objects to be fermented.

TABLE 1

| | Agitation | Agitation + Diffusion |
|---|---|---|
| Before Application (° C.) | 45° C. | 47° C. |
| After Application (° C.) | 42° C. | 39° C. |
| Amount of Temperature | 3° C. | 8° C. |
| Change (ΔT) | | |

As described above, it is possible to perform fermentation at an overall temperature in the range of 37° C. to 43° C. suitable for Bacillus, the seed microorganism, through temperature decrease by agitation. The maintenance of an appropriate temperature for fermentation is regarded as a very important control factor to prevent the adhesion of viscous materials due to the PGA component of Bacillus and to maintain the stability of enzyme reactions.

Additionally, in conjunction with this, it was possible to improve the quality of the fermentation product through oxygen supply into the objects to be fermented by the air diffusers 300. One of the most challenging and important factors in the setup of a continuous fermentation device, in particular with regard to the facilities of a continuous fermentation device for aerobic solid fermentation, is an appropriate supply of oxygen to solid objects to be fermented. Insufficient air delivery may cause deterioration in the metabolism of aerobic microorganisms, thus resulting in failure to obtain the desired quality.

In this regard, as in the continuous fermentation device according to the present disclosure, the distribution pipes 320 of the air diffusers 300 were arranged in a transport direction on a conveyor belt 110 to be suitable for the continuous fermentation device using the conveyor belt 110, and the apertures 331 were provided in the distribution pipes 320 so that the apertures 331 are optimized for each size (2 mm to 3 mm) and for each location. Additionally, for optimum oxygen supply as described above, the distribution pipes 320 were arranged in 3 to 8 rows, and the angles of the apertures 331 were adjusted according to the arrangement so that air could be evenly and forcibly ventilated into the objects to be fermented (see FIG. 6).

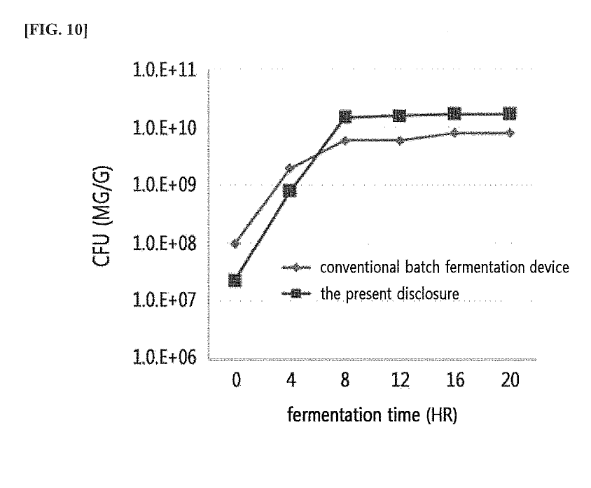

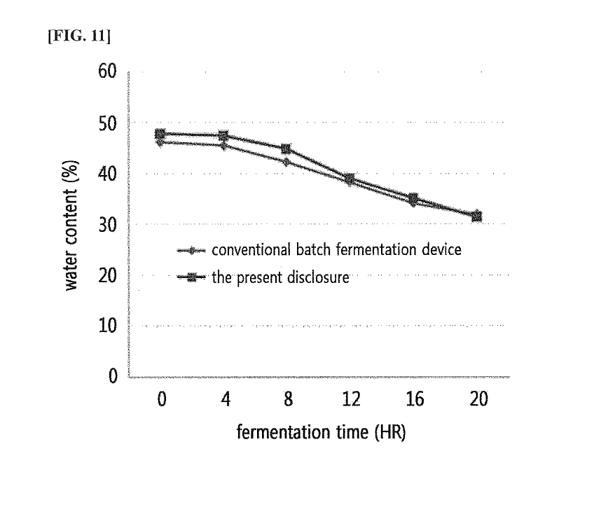

The fermentability and quality of fermentation products were evaluated using the secured air diffusers 300. As shown in FIG. 10, the growth/proliferation of the microorganism was shown to be almost identical to that in the batch fermentation device. As shown in FIG. 11, it was confirmed that the change in water content during solid fermentation was also shown to be at the same rate as in the batch fermentation device. These results show that the heat generated inside the objects to be fermented through aerobic fermentation is appropriately discharged to the outside.

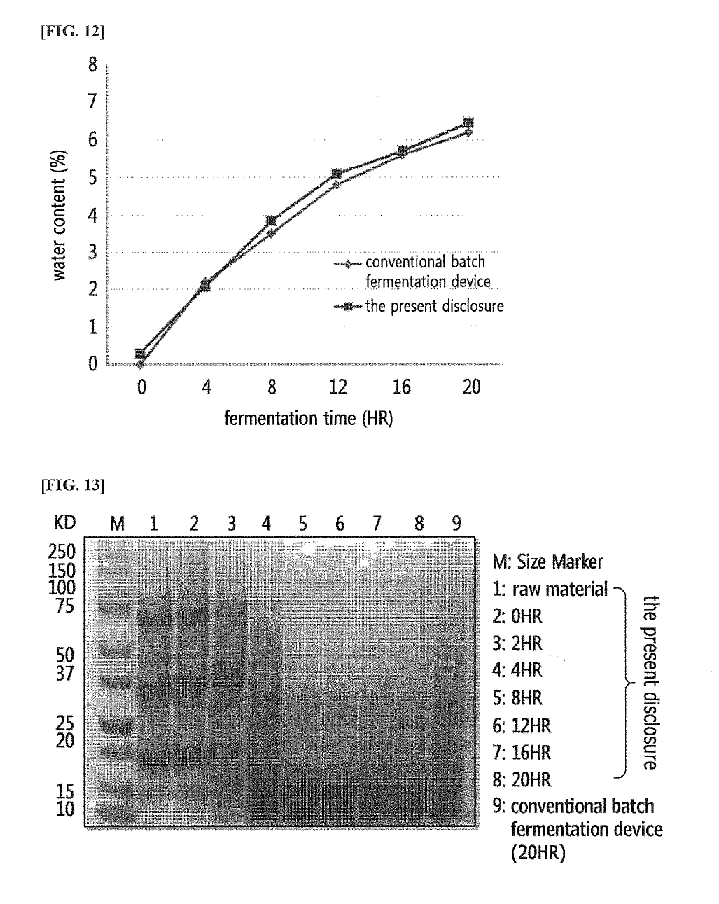

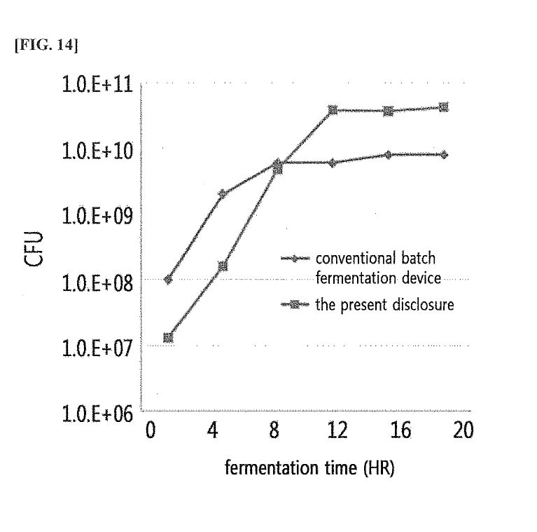

Additionally, as shown in FIG. 12, it was confirmed that the change in the crude protein content rather rapidly increased compared to that in the batch fermentation device. This indicates that, as described above, the effects of enhancing the air permeability of the objects to be fermented through the optimization of the arrangement of the distribution pipes 320 and the arrangement angles of the apertures 331 are exhibited as a change in the overall quality.

Furthermore, the main purpose of the application of the seed microorganism, Bacillus, is to induce the peptidation of proteins in soybean meal through enzymatic hydrolysis (the protease in Bacillus), and this effect improves feed digestibility, thereby improving feed nutrition efficiency.

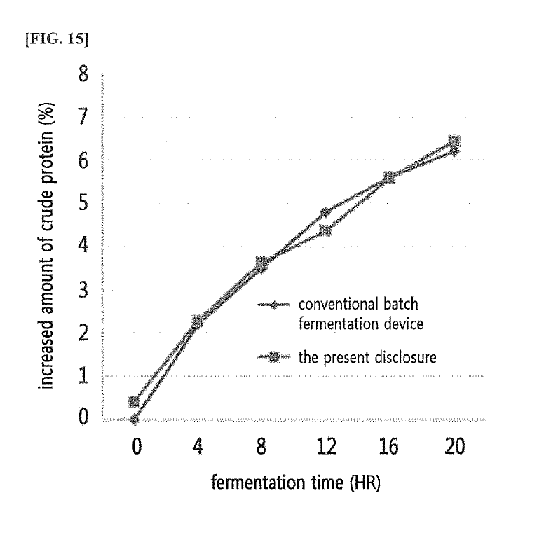

As shown in FIG. 13, it was confirmed that when fermentation was performed using the continuous fermentation device according to the present disclosure, protein degradation proceeded well with time. This result showed a protein decomposition pattern similar to that by the batch fermentation device, thus confirming that the utilization of the continuous fermentation device of the present disclosure can secure digestibility equal to or greater than that obtained when the final product quality was utilized.

Then, the activity and KOH solubility of trypsin inhibitor (i.e., a representative inhibitor of digestibility) were measured. As shown in Table 2 below, it was confirmed that the activity of trypsin inhibitor, an anti-nutritional factor, was decreased during the processes of treating raw materials, steaming, and fermentation. Additionally, the KOH solubility was also confirmed to be excellent compared to that prepared by the batch fermentation device, and the comparison of final quality revealed that the quality of the product prepared by the continuous fermentation device of the present disclosure was equal to or higher than that prepared by the batch fermentation device. From these results, it can be concluded that the Bacillus fermentation efficiency is superior in the continuous fermentation device according to the present disclosure.

TABLE 2

| | | Present | Present | Present | Present |
|---|---|---|---|---|---|
| | Batch | Disclosure | Disclosure | Disclosure | Disclosure |
| | Fermentation | (Experi- | (Experi- | (Experi- | (Experi- |
| | Device | ment 1) | ment 2) | ment 3) | ment 4) |
| Trypsin | 1.0 or less | 0.72 | 0.67 | 0.62 | 0.73 |
| Inhibitor | | | | | |
| (mg/g) | | | | | |
| KOH | 65 or greater | 77.4 | 82.3 | 81.8 | 83.5 |
| Solubility | | | | | |

Conclusively, oxygen delivery can be more effectively performed by arranging the air diffusers 300 so as to facilitate oxygen supply within the solid objects to be fermented and this will enable the maintenance of optimal fermentation conditions. Additionally, the device of the present disclosure can maximize the solid fermentation of aerobic microorganisms and ensure the quality of the fermentation product.

As described above, the results of the quality of the fermentation product prepared by the device of the present disclosure, which were equal to or greater than that obtained by the conventional batch fermentation device within the same fermentation time, were secured by enhancing the major control factors with regard to solid objects to be fermented, such as temperature of a fermentation product or air feeding into the objects to be fermented (air permeability), and this is a factor that can provide many opportunities for cost reduction by improving productivity through continuous operation and efficient installation space in the progress of industrialization. Additionally, since the amount of load for agitation in the continuous fermentation is less than that in the batch fermentation, the device for agitation can be installed at a lower cost, and the device can be configured to allow ventilation in various places for all of the objects to be fermented, thus being effective for aerobic fermentation. In addition, the device of the present disclosure has an advantage in that it is sufficiently possible to further reduce the investment cost of the facility according to the height of the objects to be fermented.

Additionally, as described above, the guide belt 120 provided on the conveyor belt 110 can prevent falling-off of the objects to be fermented and subsequent contamination thereof. However, there was a concern that surface resistance of the guides 120 may occur during fermentation if fermentation is performed after the guides 120 are provided.

However, as shown in FIG. 14, the guides 120 did not show any surface resistance during this experiment, and it was confirmed that all of the objects to be fermented were sufficient for aerobic fermentation by the air delivered by the distribution pipes 320 described above.

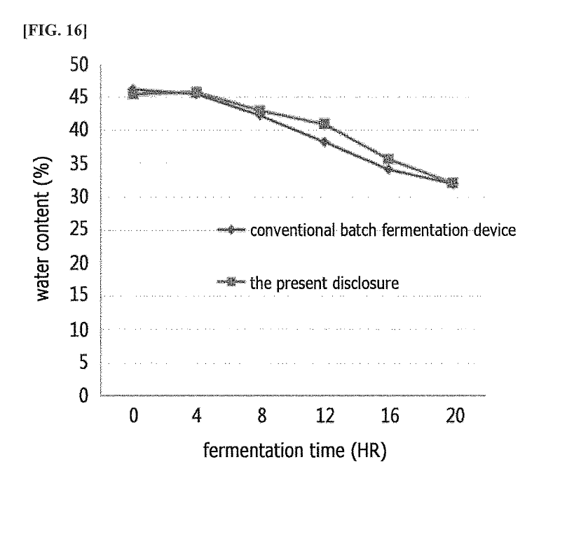

Additionally, as shown in FIGS. 15 and 16, there was also no influence by the guides 120 on the results with regard to the growth of the microorganism, increase of the protein amount, and water content in the final fermentation product of the objects to be fermented. From these results, it is possible to minimize the width of the conveyor belt 110 through the guides 120 and prevent the fermentation product from being spread and falling off after agitation.

Although the preferred embodiments of the present disclosure have been described above, the present disclosure is not limited to the specific embodiments described above. That is, one of ordinary skill in the art to which it belongs will be able to understand that numerous changes and modifications to the present disclosure are possible without departing from the spirit and scope of the appended claims, and all of such equivalents of alterations and modifications would be considered within the scope of the present disclosure.

### REFERENCE NUMERALS

100: Fermentation Module
110: Conveyor Belt
200: Agitator
300: Air Diffuser
400: Pulverizing Unit
500: Air Conditioning Device
600: Air Supply Unit

1. A continuous fermentation device comprising a plurality of fermentation modules 100, each of the plurality of fermentation modules 100 comprising:
a conveyor belt 110, which transports objects to be fermented that are continuously fed thereinto from one side to the opposite side thereof;
agitators 200, which are located on the conveyor belt 110; and
air diffusers 300, which are located on one side of the conveyor belt 110,
wherein the continuously fed objects to be fermented are fermented while being passed through the fermentation modules 100 and are continuously discharged.

2. The continuous fermentation device according to claim 1, wherein the plurality of fermentation modules 100 are in a stacked form where one side and the opposite side of each conveyor belt 110 are connected to each other, and the continuously fed objects to be fermented are continuously fermented while being passed through the plurality of fermentation modules 100.

3. The continuous fermentation device according to claim 2, wherein the plurality of conveyor belts 110 of the plurality of fermentation modules 100 form a stack of two rows, and each of the plurality of fermentation modules 100 further comprises a belt connection part 150, which connects one side and the opposite side of each of the conveyor belts 110 in each row, and the continuously fed objects to be fermented are continuously fermented while being passed through the fermentation modules 100.

4. The continuous fermentation device according to claim 1, wherein each of the agitators 200 comprises:
a shaft 210, which is located on the conveyor belt 110;
a plurality of support frames 220, wherein each support frame radially extends from one side and the opposite side of the outer circumference of the shaft 210;
connecting frames 230, wherein each connecting frame connects the ends of the support frames 220 located at each of the opposite sides of the shaft 210; and
a plurality of agitating frames 240, wherein each agitating frame connects the connecting frames 230 on one side and the opposite side.

5. The continuous fermentation device according to claim 4, wherein each of the agitators 200 agitates the objects to be fermented, which are transported on the conveyor belt 110 as each of the agitating frames 240 moves, as the shaft 210 rotates.

6. The continuous fermentation device according to claim 4, further comprising agitation wings 250, wherein each agitation wing is located on one side of the outer circumference of the plurality of agitating frames 240 in the longitudinal direction.

7. The continuous fermentation device according to claim 4, wherein the shaft 210 is height-adjustable.

8. The continuous fermentation device according to claim 1, wherein the air diffuser 300 comprises:
a main supply pipe 310, which is located across the top side of the conveyor belt 110 on one side of the conveyor belt 110; and
distribution pipes 320, wherein each distribution pipe extends from one side of the main supply pipe 310 and is located in the longitudinal direction of the top side of the conveyor belt 110, and a plurality of apertures 331 are formed around the periphery thereof,
wherein each of the distribution pipes 320 injects air inside the objects to be fermented when the objects to be fermented are transported on the conveyor belt 110.

9. The continuous fermentation device according to claim 8, wherein the apertures 331 are formed in at least two rows and the objects to be fermented are supplied with air through the apertures 331.

10. The continuous fermentation device according to claim 9, wherein each row formed by the apertures 331 is arranged at a predetermined angle from the center of the distribution pipes 320.

11. The continuous fermentation device according to claim 9, wherein the apertures 331 are arranged in three rows on one side of the outer circumference of the distribution pipes 320, wherein each row formed by the apertures 331 is arranged with an angular difference of from 60 degrees or greater to 90 degrees or less from the center of the distribution pipes 320.

12. The continuous fermentation device according to claim 1, further comprising guides 120, which have a predetermined height and are located in the longitudinal direction on both sides of the conveyor belt 110.

13. The continuous fermentation device according to claim 1, further comprising a conveyor belt 110′ which transports the objects to be fermented that are continuously fermented and discharged to a drying unit.

14. The continuous fermentation device according to claim 1, further comprising:
a plurality of air conditioning devices 500, which are provided on one side of each of the conveyor belt 100 for suction of odors generated during the fermentation process of the objects to be fermented; and
odor-processing units 60, which are connected to the plurality of air conditioning devices 500,
wherein the odors sucked from the plurality of air conditioning devices 500 are integrated and processed in the odor-processing units 60.
